(19) United States
(12) Patent Application Publication
(10) Pub. No.: US 2025/0152209 A1
(43) Pub. Date: May 15, 2025

(54) LATERAL MASS FIXATION SYSTEM

(71) Applicant: PROVIDENCE MEDICAL TECHNOLOGY, INC., Pleasanton, CA (US)

(72) Inventors: Bruce M. McCormack, San Francisco, CA (US); Edward Liou, Pleasanton, CA (US); Shigeru Tanaka, Half Moon Bay, CA (US); Christopher U. Phan, Dublin, CA (US)

(21) Appl. No.: 19/028,144

(22) Filed: Jan. 17, 2025

Related U.S. Application Data
(63) Continuation of application No. 17/373,120, filed on Jul. 12, 2021.
Application No. 17/373,120 is a continuation of application No. 16/272,180, filed on Feb. 11, 2019, now Pat. No. 11,058,466.
Application No. 16/272,180 is a continuation of application No. 14/723,410, filed on May 27, 2015, now Pat. No. 10,201,375.
(60) Provisional application No. 62/004,143, filed on May 28, 2014.

(51) Int. Cl. A61B 17/70; A61B 17/16; A61B 17/17

(52) U.S. Cl. CPC A61B 17/7082; A61B 17/1671; A61B 17/1757; A61B 17/7011; A61B 17/7032; A61B 17/1604; A61B 17/7064

(57) ABSTRACT

A device for accessing and guiding at least one fixation device to a spine may include a distal portion configured to fit in a facet of the spine and a proximal portion extending from the distal portion. The proximal portion may be detachable from the distal portion and may be hollow or solid. A system for accessing and guiding at least one fixation device to a spine may include a distal portion configured to fit in a facet of the spine, a proximal portion extending from the distal portion, and a slidable guide device for sliding over the facet guide device to guide at least one instrument to the spine.

### CROSS REFERENCE TO RELATED APPLICATIONS

This application is a continuation of U.S. patent application Ser. No. 17/373,120, filed Jul. 12, 2021, which is a continuation of U.S. patent application Ser. No. 16/272,180, filed Feb. 11, 2019, now U.S. Pat. No. 11,058,466, issued Jul. 13, 2021, which is a continuation of U.S. patent application Ser. No. 14/723,410, filed May 27, 2015, now U.S. Pat. No. 10,201,375, issued Feb. 12, 2019, which claims priority to U.S. Provisional Patent Application No. 62/004, 143, filed May 28, 2014. The full disclosures of the above-listed patent applications are hereby incorporated by reference herein in their entireties and for any purpose.

### BACKGROUND

Posterior cervical fusion with lateral mass fixation is the most rigid cervical instrumentation. It requires extensive dissection of muscles and ligaments off the posterior spine, so that the surgeon can have direct visualization to safely perform the procedure. This dissection causes acute and chronic soft tissue pain syndrome. Acutely, patients are typically hospitalized for three to four days for pain control that requires IV narcotics. This is compared to one-day hospitalization for anterior approaches that do not require any muscle or soft tissue dissection. Long-term patients with posterior approaches frequently have persistent pain due to the extensive nature of the dissection. Sometimes, after posterior-access cervical fusion surgery, soft tissues may not return to anatomic position and may be permanently deformed. Persistent pain after posterior surgical approaches is referred to as post-laminectomy syndrome. (FIG. 1 is a lateral view of the C5 and C6 cervical vertebrae, illustrating the anatomy.)

Therefore, since it is considered less traumatic to the patient compared to posterior approaches, anterior cervical spinal fusion surgery has generally been preferred over posterior fusion surgery. At the same time, posterior approaches to the cervical spine do have some advantages over anterior approaches.

Lateral mass or pedicle screw fixation provides more rigid fixation of the cervical spine than anterior plates, interbody devices and interspinous wiring. It is best for traumatic instability, but it has also been used for degenerative conditions. Despite being the best fixation, lateral mass fixation is often avoided, because of the morbidity of the soft tissue dissection, as noted above. (FIGS. 2A and 2B are posterior and lateral views, respectively, of a cervical spine with posterior fixation devices applied thereto.)

Starting a drill hole or inserting a screw into a lateral mass of a vertebra cannot currently be accomplished using a percutaneous approach. This is because soft tissue gets caught up in the drill, and the drill can skid off the bone and go out of control. Awls and firm pressure placed on bone with screws without direct visualization is dangerous in the posterior cervical spine, unless the surgeon has removed soft tissue and has direct visualization.

Therefore, it would be advantageous to have improved devices, systems and methods for performing cervical spinal fusion procedures via posterior access approaches. Ideally, these devices, systems and methods would allow for minimally invasive or less invasive access and fixation, as well as helping ensure proper placement of the fixation devices. At least some of these objectives will be met by the embodiments described herein.

### BRIEF SUMMARY

The various embodiments described herein provide devices, systems and methods for accessing the cervical spine via a posterior approach and implanting a spinal fixation device in the cervical spine. The embodiments described below generally include a guide device, through which or along which one or more spinal fixation devices may be advanced. The guide devices described herein generally include a distal end that can be inserted into a cervical facet. Once inserted into a facet, the guide device is relatively stabilized (or “docked”) on the spine and thus can be used as a point of stabilization.

A device for accessing and guiding at least one fixation device to a spine is disclosed. In some aspects, the device includes a distal portion configured to fit in a facet of the spine and a proximal portion extending from the distal portion. In various embodiments, the distal and proximal portions are hollow. In some embodiments, the distal and proximal portions are solid. The distal portion may be removable from the proximal portion. In some embodiments, the distal portion includes a chamfered or beveled end portion configured to facilitate insertion of the distal portion in the facet of the spine. The proximal portion may include a slot formed therethrough for receiving and advancing a fixation device to the spine. The end of the proximal portion may include opposing sides having a concave shape and/or opposing sides having a convex shape.

A system for accessing and guiding at least one fixation device to a spine is disclosed. In one aspect, the system includes a facet guide device, the facet guide device including a distal portion configured to fit in a facet of the spine and a proximal portion extending from the distal portion. The system further includes a slidable guide device for sliding over the facet guide device to guide at least one instrument to the spine. The slidable guide device may be rotatable about a longitudinal axis of the facet guide device. The instrument may be a decortication device. The slidable guide device may be a double-barreled or dual-lumen guide tube. The slidable guide device may further include a drill guide having at least one drill path defined therein. The proximal portion of the facet guide device may have one of a circular cross-sectional shape or a square cross-sectional shape. The proximal portion of the facet guide device may have opposing sides having a concave shape. The proximal portion of the facet guide device may have opposing sides having a convex shape. The slidable guide device may include a first tube for sliding over the proximal portion of the facet guide device and a second tube mounted on a side of the first tube for guiding the at least one instrument. The system may further include at least one bone screw for advancing through the slideable guide device.

A method for implanting a spinal fixation implant is disclosed. The method includes advancing a guide device into a facet between two adjacent vertebrae, advancing a fixation device along the guide device, and attaching the fixation device to at least one of the two adjacent vertebrae. The method may further include attaching the fixation device by one of attaching a plate to a facet implant located in the facet or attaching a plate to the two adjacent vertebrae.

These and other aspects and embodiments will be described in further detail below, in reference to the attached drawing figures.

### BRIEF DESCRIPTION OF THE DRAWINGS

FIG. 1 is a lateral view of the C5 and C6 cervical vertebrae, illustrating the anatomy.

FIGS. 2A and 2B are posterior and lateral views, respectively, of a cervical spine with prior art posterior fixation devices applied thereto.

FIGS. 3A and 3B are posterior views of a portion of a cervical spine, illustrating insertion of a distal portion of a guide device into a facet between two cervical vertebrae, according to one embodiment.

FIGS. 4A and 4B are posterior views of a portion of a cervical spine, illustrating insertion of a distal portion of a guide device into a facet between two cervical vertebrae, according to an embodiment.

FIGS. 5A and 5B are lateral views of a portion of a cervical spine, illustrating insertion of a distal portion of a guide device into a facet between two cervical vertebrae and removal of a proximal portion of the guide device from the distal portion, according to one embodiment.

FIGS. 6A-6D are perspective views of a portion of a cervical spine, illustrating a system and method for inserting a lateral mass implant, according to one embodiment.

FIGS. 7A and 7B are perspective views of a portion of a cervical spine, illustrating a system and method for inserting a lateral mass implant, according to an embodiment.

FIGS. 8A-8E are various views of a portion of a cervical spine, illustrating a system and method for inserting a lateral mass implant, according to an embodiment.

FIGS. 9A-9C are various views of a portion of a cervical spine, illustrating a system and method for inserting a lateral mass implant, according to an embodiment.

FIGS. 10A and 10B are perspective views of a portion of a cervical spine, illustrating a system and method for advancing a decorticator device over a guide device, according to one embodiment.

FIGS. 11A-11D are perspective views of a portion of a cervical spine, illustrating a system and method for advancing a drill through a guide device, according to one embodiment.

FIG. 12 is a perspective view of a portion of a cervical spine, illustrating a system and method for inserting a lateral mass implant, according to an embodiment.

FIGS. 13A-13N illustrate various views of a portion of a cervical spine, illustrating a system and method for inserting a lateral mass implant, according to an embodiment.

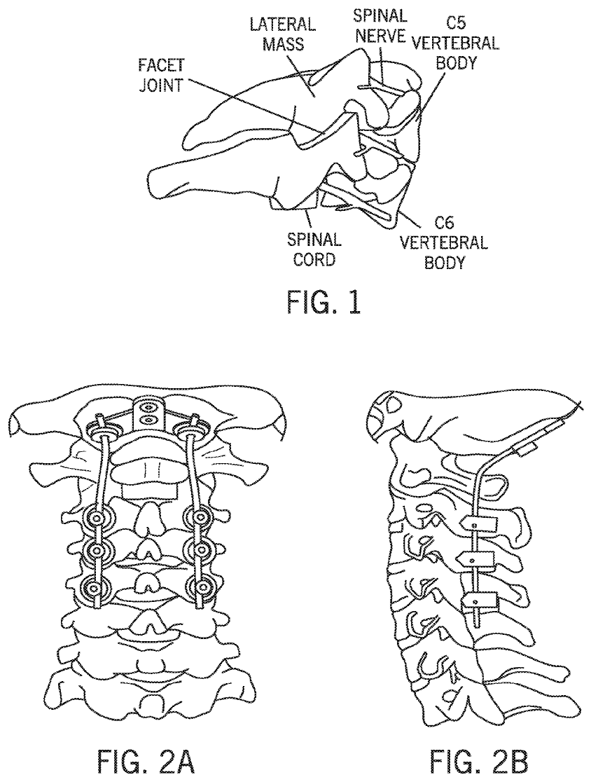

### DETAILED DESCRIPTION

The various embodiments described herein provide devices, systems and methods for accessing the cervical spine via a posterior approach and implanting a spinal fixation device in the cervical spine. The embodiments allow for a posterior approach using minimally invasive or less invasive techniques. The embodiments described below generally include a guide tool, through which or along which one or more spinal fixation devices may be advanced.

The surgeon may advance the guide tool into the facet from outside the patient though a minimally invasive or less invasive incision, and then may hold the guide tool via a handle or proximal end residing outside the patient. This fixed point deep in the spine can be used to advance drills, awls, plates, rods and screws, to instrument the posterior cervical spine other than the facet, from a percutaneous approach without direct visualization. This avoids stripping all the soft tissue off the spine. A fixed point deep in the patient's spine prevents instruments from slipping off the spine or drills catching soft tissue and skidding out of control. Also, the cervical facet has a fixed anatomic relationship to lateral mass bone consistent in all patients. Instruments can be advanced over the facet tool to reliable landmarks on the lateral mass without direct visualization.

Some of the devices, systems and methods described herein may include, be performed using, or be similar to one or more components of the DTRAX® Spinal System, from Providence Medical Technology, Inc. (www.providencemt.com). Various components of the DTRAX® Spinal System may be modified or adjusted, according to various embodiments, for uses described herein.

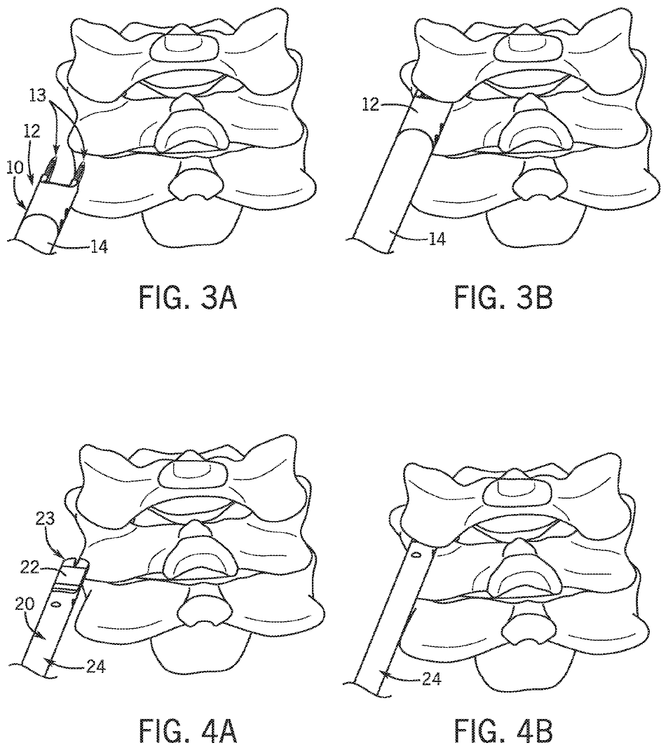

Referring now to FIGS. 3A and 3B, in one embodiment, a facet guide tool or device 10 may include a distal portion 12 configured for insertion into a facet space between two cervical vertebrae and a proximal portion 14 (or “shaft”) that extends proximally from the distal portion 12. The proximal shaft portion 14 is generally long enough to extend from the distal portion 12 to a location outside the patient, where it can be held and manipulated by the surgeon. In one embodiment, the distal portion 12 may include two tines 13. In various embodiments, the distal portion 12 and the proximal portion 14 may either be two attached pieces or may be one piece (e.g. monolithically formed or integrally formed). In some embodiments, the two attached pieces may be detachable, as will be described further below. In some embodiments, the distal portion 12 may be temporary and may be removed once lateral mass fixation is achieved. The distal portion 12 is generally sized and shaped to fit snugly into the facet and abut the pedicle. The tight fit of distal portion 12 in the facet, due to forces applied by ligaments surrounding the area, helps provide stability to the facet guide tool 10 while fixation devices are advanced to the site.

In the embodiment of FIGS. 3A and 3B, the distal portion 12 and proximal portion 14 are hollow, thus forming a central lumen or bore (not visible in the figures), through which one or more facet fixation devices may be advanced. Alternatively or additionally, one or more fixation devices may be advanced over the guide tool 10 to the cervical spine. For example, a fixation device may have a hole formed therethrough of complementary size and shape to the guide tool 10. The hole may be aligned with the guide tool 10 and the fixation device may be advanced along the guide tool.

With reference to FIGS. 4A and 4B, in another embodiment, a facet guide tool or device 20 may include a distal portion 22 and a proximal portion 24. The distal portion 22 may include a beveled edge 23 to aid insertion of the distal portion into a facet. The distal portion 22 and the proximal portion 24 may both be solid, rather than hollow. In this embodiment, the guide tool 20 acts as a rail, over which one or more fixation devices or other devices may be advanced. In use, the guide tool 20 may be inserted in one facet or in multiple facets during a procedure. If used in multiple facets, multiple guide tools 20 may be inserted simultaneously, or the same guide tool may be inserted sequentially into multiple facets, to implant lateral mass or pedicle screw spinal instrumentation from a percutaneous approach. According to various alternative embodiments, the proximal shaft portion 24 may be flexible or rigid. Its purpose is to extend to the skin surface and serve as a guide for drills, plates, rods, screws and/or other tools of spinal instrumentation.

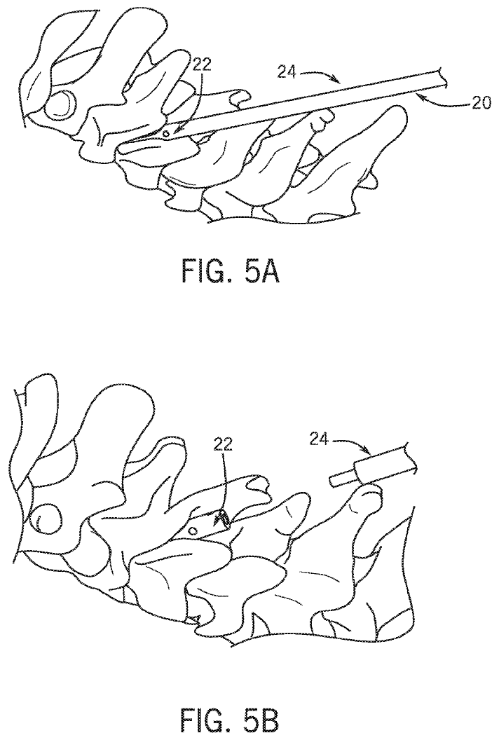

Referring now to FIGS. 5A and 5B, in some embodiments, the distal portion 22 of the guide tool or device 20 may be removable from the proximal portion 24, so that the distal portion 22 may be left in the facet as an implant. In some embodiments, one or more lateral mass fixation devices may then be attached to the distal portion 22 for contacting and attaching to lateral masses of adjacent vertebrae.

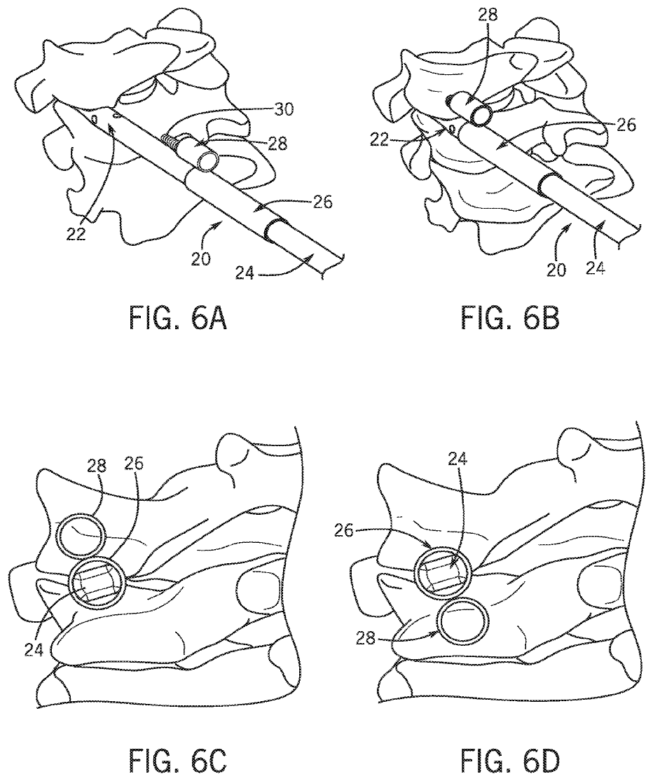

With reference to FIGS. 6A-6D, in one embodiment, a system for accessing and attaching fixation devices to a cervical spine facet may include the guide tool or device 20 with distal portion 22 and proximal portion 24, as described above. The system may also include an outer, sliding guide tube 26 and a side-mounted guide member 28 attached to the guide tube 26. A screw 30 may be advanced through the side-mounted guide member 28 for attachment to bone. As illustrated in FIGS. 6C and 6D, sliding guide tube 26 may be rotated about the proximal portion 24 of the guide device 20, to change the position of the side-mounted guide member 28. This change of position may be used, for example, to attach two screws to two adjacent vertebrae. The proximal portion 24 may have different cross-sectional shapes in different embodiments, with the circular cross-sectional shape providing 360-degree rotation of instruments advanced over it.

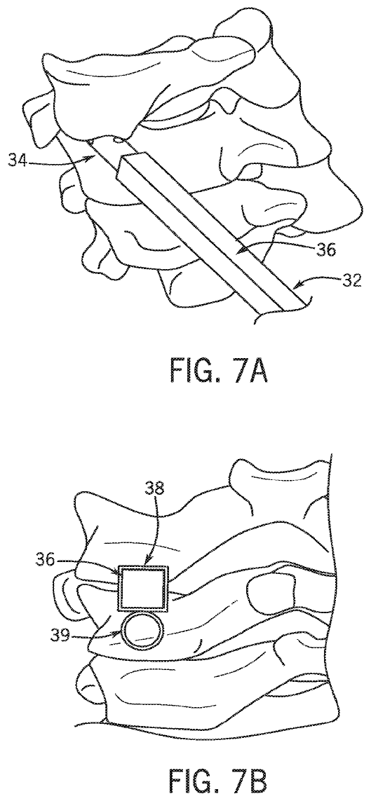

Referring to FIGS. 7A and 7B, in an alternative embodiment, a facet guide tool or device 32 may include a distal portion 34 and a proximal shaft portion 36 having a square cross-sectional shape. As illustrated in FIG. 7B, an additional guide device 38 may be advanced over the proximal portion 36 and may include a side-mounted guide tube 39. In this embodiment, the square cross-sectional shape of the proximal portion 36 allows instruments to be advanced at a fixed 90 degree angle to the facet surface. This may be useful for lateral mass fixation, because the typical screw fixation is at the midpoint of the lateral mass, which is immediately above the midpoint of the facet.

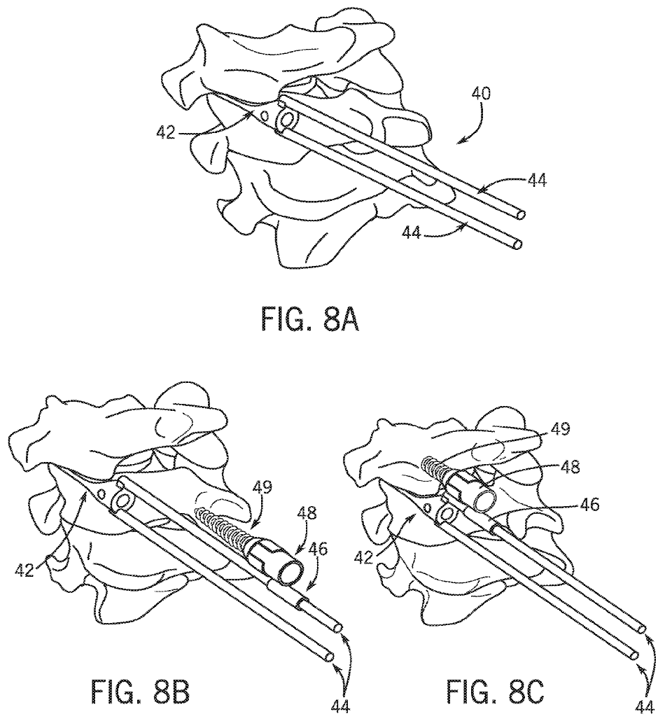

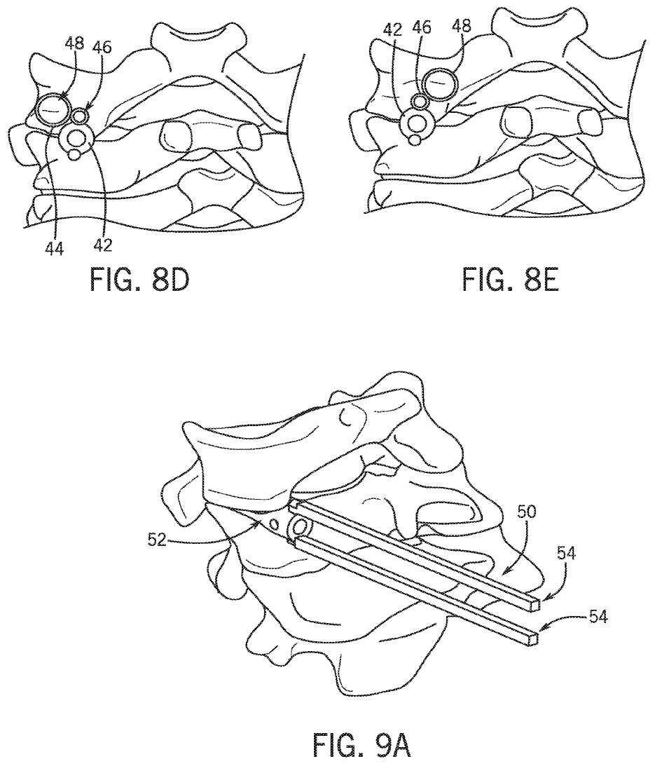

In yet another embodiment, and with reference now to FIGS. 8A-8E, a facet guide tool 40 may include a distal portion 42 and two or more proximal shafts 44. A guide system may further include a slidable guide instrument 46, with a side-mounted guide 48, which may be used to advance a screw 49 into bone. The proximal shafts 44 may be advantageous, for example, in advancing multiple guide instruments 46 to the cervical spine, either simultaneously or sequentially, for attaching screws to adjacent vertebrae. As illustrated in FIGS. 8D and 8E, the guide instrument 46 may also be rotated over one of the proximal shafts 44 to change the position of the side-mounted guide 48 relative to bone.

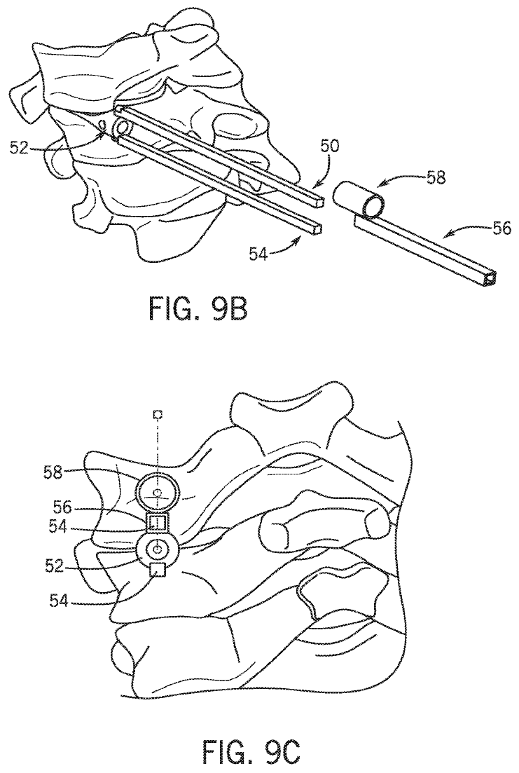

FIGS. 9A-9C illustrate yet another embodiment of a facet guide tool or device 50. Similar to the previously described embodiment, in this embodiment, the guide tool 50 includes a distal portion 52 and two or more proximal shafts 54. A guide system may further include a slidable guide instrument 56, with a side-mounted guide 58, which may be used to advance a screw (not shown) into bone. In this embodiment, the proximal shafts 54 have a square cross-sectional shape. As described above, the square cross-sectional shape may be used to orient the guide instrument 56 at 90-degree increments.

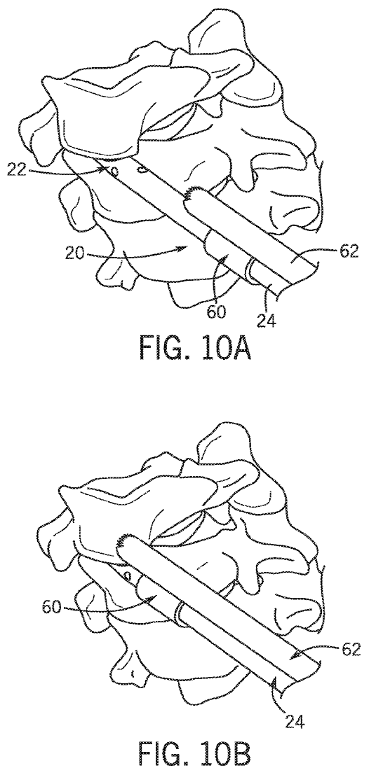

FIGS. 10A and 10B illustrate another instrument that may be advanced over a facet guide tool or device 20. In this embodiment, a slidable guide tube 60 with a side-mounted decortication device 62 is shown advancing over the guide tool 20. The decortication tool 62 may be used to cut or decorticate vertebral bone, as part of a fixation procedure. Various embodiments may include this and/or any other similar instrumentation, such as but not limited to screws, staples, posts in the lateral masses, and/or the like. Additional instrumentation, such as a rod or plate, may also be advanced over the facet guide tool 20. Plates generally act as tension bands to connect the rostral and caudal facet and serves to limit flexion and extension as well as lateral bending.

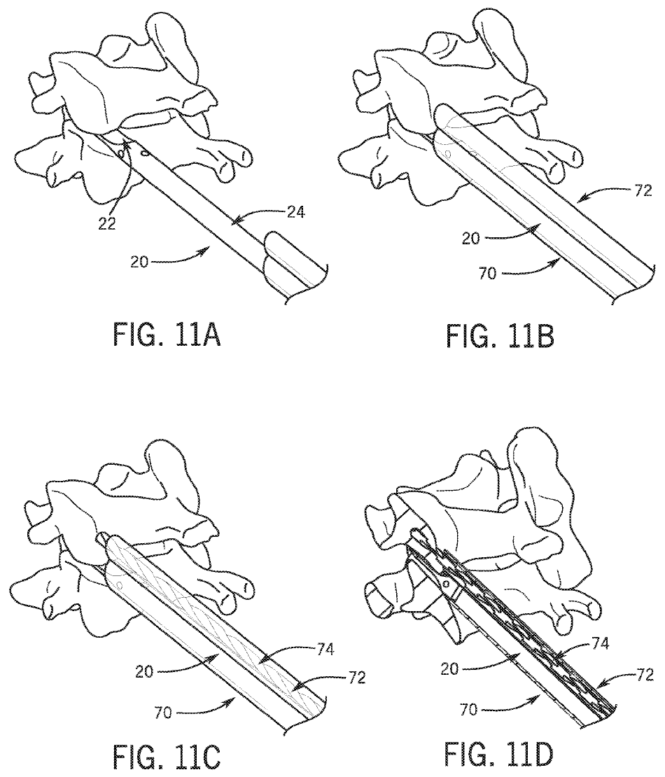

Referring now to FIGS. 11A-11D, in another embodiment, the facet guide tool or device 20 may be used to advance a double-barreled or dual-lumen guide tube device 70, having a side-mounted tube 72, to the cervical spine. As illustrated in FIGS. 11C and 11D, in one embodiment, a drill 74 may be advanced through the side-mounted tube. This guide tube device 70 thus allows for drilling of the lateral mass at the same angle as the facet.

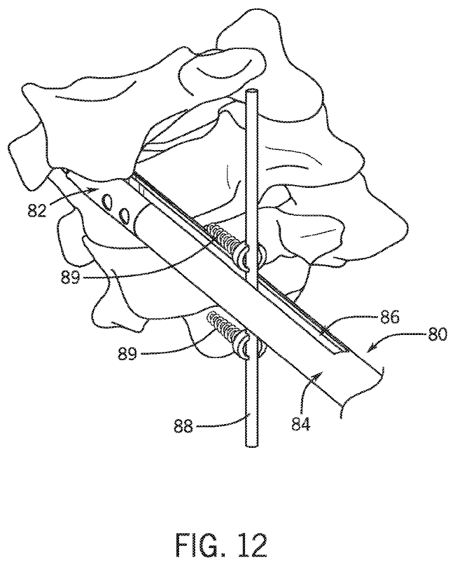

In yet another embodiment, and with reference to FIG. 12, a facet guide tool 80 may have a distal portion 82 and a proximal shaft portion 84, including a slot 86. The slot 86 may be used for advancing a fixation device, such as rod 88 (or plate) and screws 89 for attachment to the rostral and caudal lateral mass.

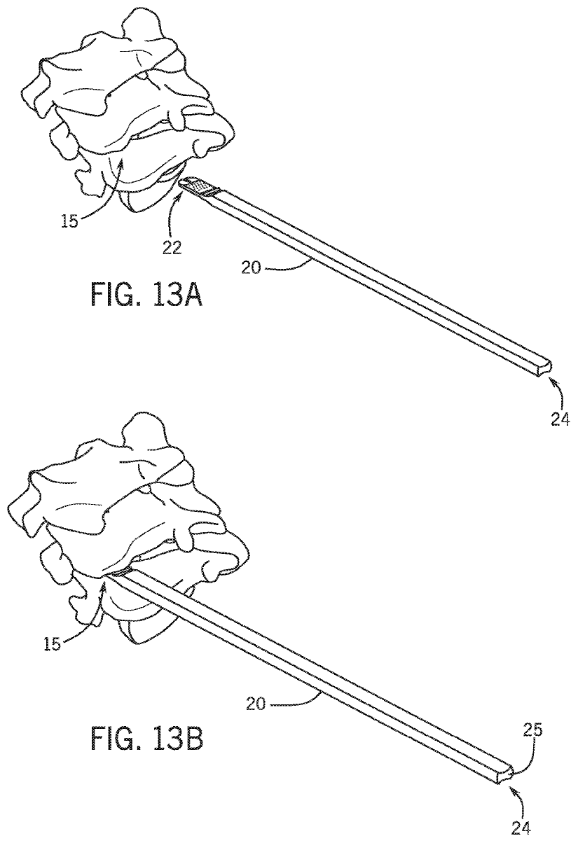

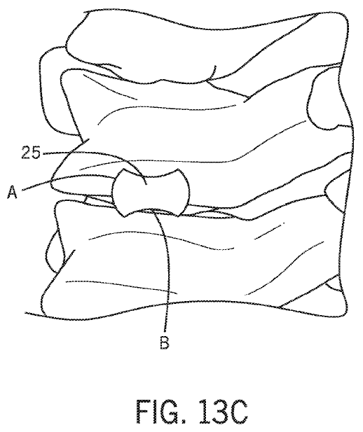

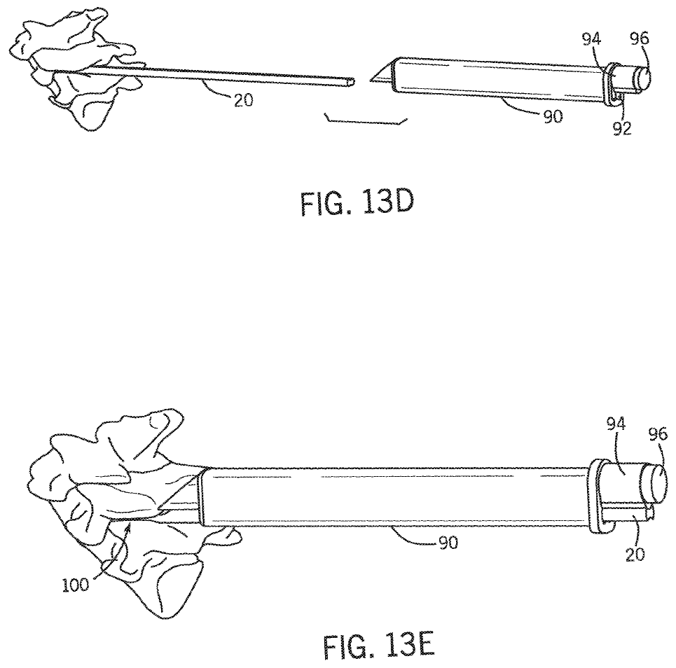

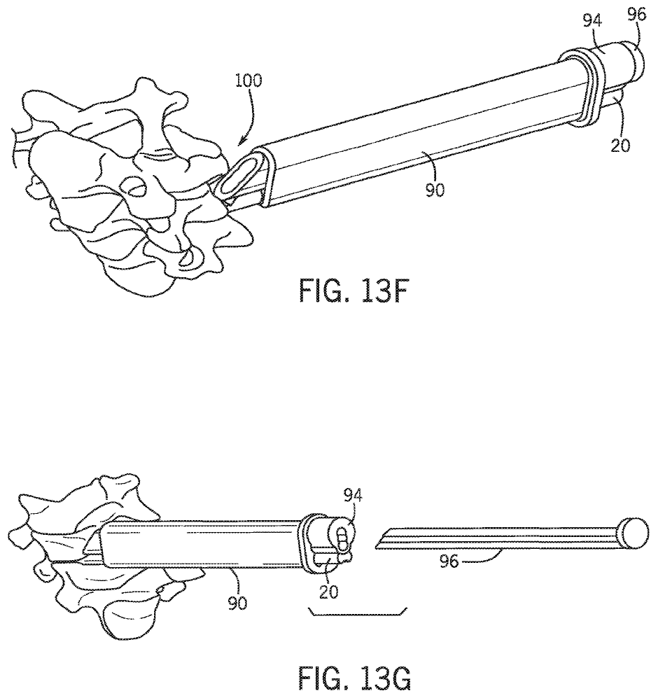

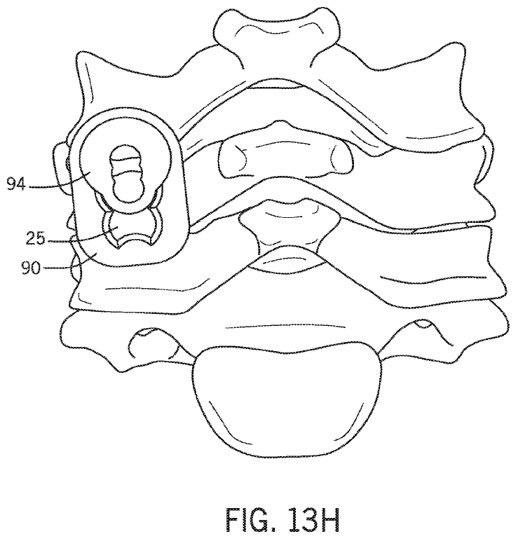

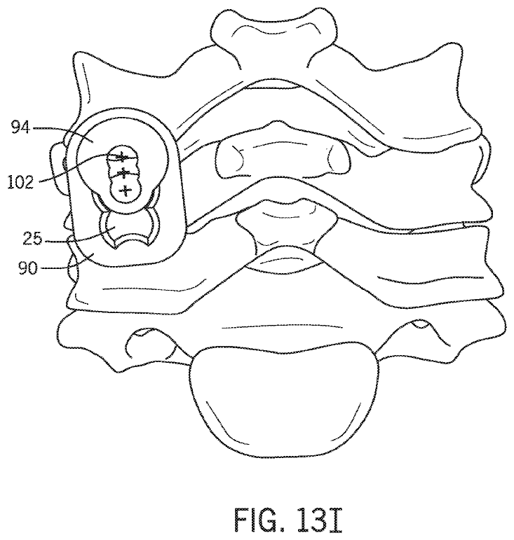

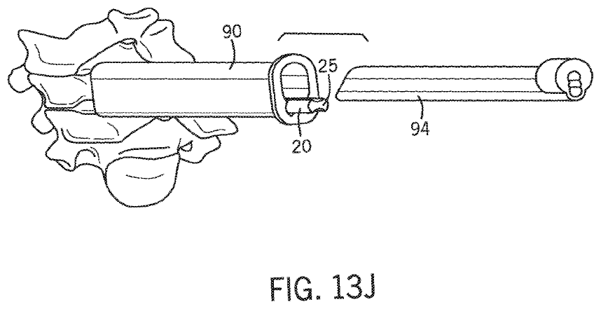

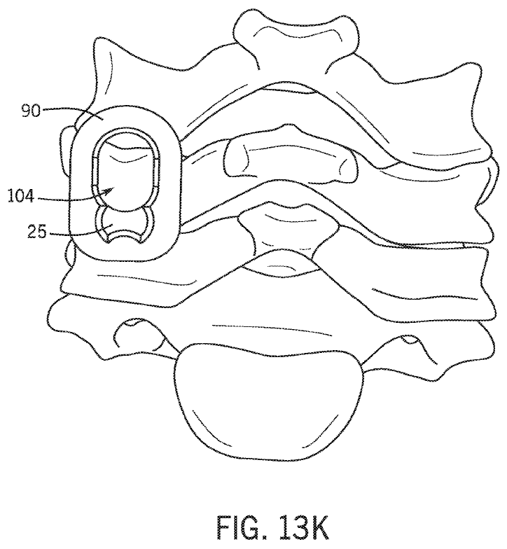

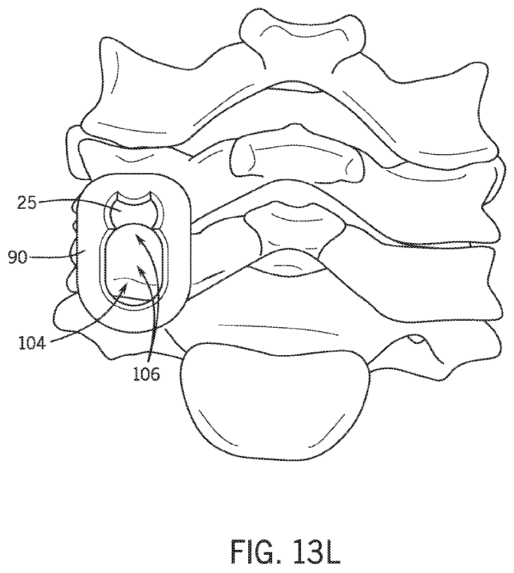

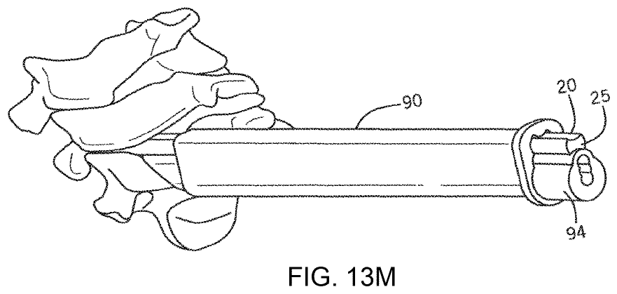

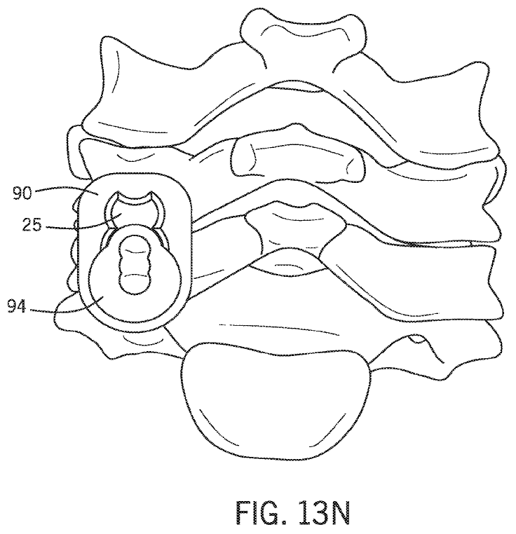

With reference to FIGS. 13A-13N, in one embodiment, a system for accessing and attaching fixation devices to a cervical spine facet 15 may include the guide tool 20 with distal portion 22 and proximal portion 24, as described above (see FIGS. 13A-13B). FIG. 13C illustrates a proximal end 25 of the tool 20 having opposing sides with a concave shape (A) and a convex shape (B). This shaped proximal end helps to maximize accessible lateral mass area and to lock the rotational position of a sliding guide tube 90 shown in later figures. As indicated in FIG. 13D and others, the system may also include an outer, sliding guide tube 90 defining a dual-lumen 92 for receiving both a drill guide 94 and stylet 96 and the guide tool 20.

In use, and as shown in FIGS. 13D-13F, the sliding guide tube 90 may be positioned over and slid onto the proximal portion 24 of the guide device 20, and docked or otherwise stabilized on or at the superior lateral mass 100. The stylet 96 may be removed from the drill guide 94 (FIG. 13G). FIG. 13H depicts an end view of the guide tool 20 and the drill guide 94 within the dual lumen 92 of the sliding guide tube 90. As discussed above, the shape of the tool 20 limits rotational movement of the guide tube 90. As illustrated in FIG. 131, the drill guide 94 provides one or more guide paths 102 through which a drill, such as drill 74 in FIG. 11C, may be advanced through the guide 94. This guide tube device 90 thus allows for drilling of the lateral mass at or about the same angle as the facet. After drilling the pilot hole for a lateral mass screw, the drill guide 94 is removed from the guide tube 90 (FIG. 13J). As shown in FIG. 13K, a first lumen 104 of the dual lumen tube 90 now provides an opening through which a lateral mass screw (not shown) may be guided for insertion in the pre-drilled location. In some embodiments, a secondary guide tube could be used in the lumen 104 to more precisely guiding the screw to the pre-drilled location.

FIGS. 13L-13N illustrate the guide tool 20, sliding guide tube 90 and the drill guide 94 inserted in an opposite or rotated orientation (e.g. rotated 180° about the longitudinal axis of the guide tool 20) for use and screw insertion in the inferior lateral mass 106.

The C7 and T1 and T2 facets have a fixed relationship to the pedicle. All of the above devices, systems and methods may be used to cannulate the pedicle percutaneously, similar to that described for the lateral mass.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

Although the invention has been disclosed in the context of certain embodiments and examples, the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

What is claimed is:

1. A device for accessing and guiding at least one fixation device to a spine, the device comprising:
a distal portion configured to fit in a facet of the spine; and
a proximal portion extending from the distal portion.
